(19) United States
(12) Patent Application Publication
(10) Pub. No.: US 2025/0002444 A1
(43) Pub. Date: Jan. 2, 2025

(54) PROCESS FOR FORMING ALKYL ESTER OF LEVULINIC ACID

(71) Applicant: BASF SE, Ludwigshafen Am Rhein (DE)

(72) Inventors: Steffen MADER, Ludwigshafen (DE); Sven REINING, Ludwigshafen (DE); Ralf BOEHLING, Ludwigshafen (DE); Veit STEGMANN, Ludwigshafen (DE); Dorothea STARCK, Ludwighshafen (DE)

(21) Appl. No.: 18/703,748

(22) PCT Filed: Nov. 24, 2022
(86) PCT No.: PCT/EP2022/083103

§ 371 Date: Apr. 23, 2024

(30) Foreign Application Priority Data
Nov. 26, 2021 (EP) 21210844.3

(51) Int. Cl. C07C 67/40; B01J 19/00; B01J 19/02

(52) U.S. Cl. CPC C07C 67/40; B01J 19/0013; B01J 19/02; B01J 2219/00033; B01J 2219/00054; B01J 2219/00166; B01J 2219/00186; B01J 2219/0286

(57) ABSTRACT

Disclosed herein are a process for forming an alkyl ester of levulinic acid or one or more reaction products obtained by chemically converting the alkyl ester of levulinic acid, a and a method of using a pressurized continuous flow reactor system for forming one or more alkyl esters of levulinic acid in the process.

The present application relates to a process for forming alkyl ester of levulinic acid or one or more reaction products thereof (i.e. reaction products obtained by chemically converting said alkyl ester of levulinic acid), to the use of a pressurized continuous flow reactor system for forming one or more alkyl esters of levulinic acid in said process, and to an apparatus for carrying out said process.

The project leading to this application has received funding from the Bio Based Industries Joint Undertaking under the European Union's Horizon 2020 research and innovation programme under grant agreement No 720695.

As used herein, the term alkyl ester of levulinic acid denotes any individual alkyl ester of levulinic acid as well as mixtures of two or more different alkyl esters of levulinic acid. Alkyl ester of levulinic acid are industrially relevant solvents or intermediates e.g. for the manufacture of pesticides, plasticizers, polymers, cosmetics, food additives or fuel additives. Important reaction products obtainable by chemically converting alkyl ester of levulinic acid include e.g. levulinic acid, ketals of alkyl ester of levulinic acid, 1,4-pentanediol, gamma-valerolactone, alpha-angelicalactone, beta-angelicalactone, 2-methyl-tetrahydrofuran, 4-hydroxy-pentanal, 5-methyl-2-hydroxy-tetrahydrofuran, pent-4-en-1-ol, 1-pentanol, 4-pentanol, pent-4-en-al, pentanal (valeraldehyde), pentanoic acid (valeric acid) and alkyl esters of pentanoic acid.

Due to the finite nature and instability of fossil feedstock supply and for environmental reasons, replacement of fossil feed-stock by non-fossil feedstock, i.e. feedstock obtained from renewable resources like biomass, becomes more and more important.

Preparation of alkyl esters of levulinic acid from furfuryl alcohol opens a pathway for obtaining industrially-relevant intermediates, products and solvents, e.g. γ-valerolactone (GVL), 1-methyl-1,4-butanediol (MeBDO) and 2-methyltetrahydrofuran (2-MTH), from renewable sources like hemicellulosic and lignocellulosic biomass, i.e. non-fossil feedstock.

Preparation of alkyl esters of levulinic acid from furfuryl alcohol may be carried out as an acid-catalyzed reaction. Unfortunately, the acid-catalyzed reaction of furfuryl alcohol with an aliphatic alcohol to alkyl esters of levulinic acid is competed by the homopolymerization of furfuryl alcohol which is acid-catalyzed, too. Homopolymerization of furfuryl alcohol is irreversible and results in loss of furfuryl alcohol and in formation of polymeric sediments which may damage the process equipment.

U.S. Pat. No. 2,763,665 A discloses a process for the manufacture of levulinic acid esters, which comprises heating furfuryl alcohol with a different alcohol containing from 1 to 10 carbon atoms in the presence of a catalyst selected from hydrogen chloride and hydrogen bromide.

Reddy et al., Catalysis Letters, vol. 148, no. 6, 31 Mar. 2018, pp. 1731-1738, describe continuous synthesis of alkyl levulinates via alcoholysis of furfuryl alcohol using silica supported solid acid catalysts containing oxides of aluminium, tungsten, zirconium and titanium, i.e. non-protic acids.

CN105884616A discloses continuous preparation of methyl levulinate by a two-step furfural process.

U.S. Pat. No. 8,389,749 B2 describes a catalytic process for converting biomass to furan derivatives (e.g., furfural, furfuryl alcohol) using a biphasic reactor containing a reactive aqueous phase and an organic extracting phase containing an alkylphenol.

EP 3 954 676 A1 (published after the priority date of the present application) discloses to a process for the conversion of furfuryl alcohol into levulinate ester comprising contacting furfuryl alcohol; an alcohol, or a mixture thereof; and a homogeneous catalyst at a first reaction temperature in the range of from 120 to 180° C. to form a reaction mixture; and forming the levulinate ester in the reaction mixture, characterised in that the first homogeneous catalyst is a sulfonic acid catalyst.

WO 2010/102203 A1 describes a process for the conversion of furfuryl alcohol into levulinate esters in a single step reaction comprising addition of the product levulinate ester to the reaction mixture of an alkanol and furfuryl alcohol in the presence of a strong protic acid catalyst, wherein high yield of the levulinate ester is said to be accompanied by low amounts of tarry residue that do not precipitate or solidify in the reaction mixture. Thus, the reaction is carried out using the final product alkyl levulinate as the solvent in the reactor vessel. The process according to WO 2010/102203 A1 comprises contacting
a first mixture comprising an alkanol, a protic acid catalyst, e.g. sulfuric acid, and an alkyl levulinate wherein the alkyl group is the same as the alkyl group of the alkanol
and a second mixture comprising furfuryl alcohol, and an additional amount of the alkanol of the first mixture;

and forming the alkyl levulinate in the reaction mixture wherein the alkyl group is the same as the alkyl group of the alkanol. The molar ratio of alkanol to alkyl levulinate in the first mixture is about 1:20 to 1:1. About 1*10⁻⁴ to 2.5*10⁻² moles of sulfuric acid is added to the first mixture per mole of furfuryl alcohol provided in the second mixture. Due to the need to use a high excess of alkyl levulinate as solvent, the yield of said process is limited.

WO 2018/112776 A1 discloses a process for synthesizing at least one levulinate ester, said process comprising the reaction of furfuryl alcohol with at least one other alcohol in the presence of water and at least one catalyst, said furfuryl alcohol being present in a quantity of a least 5% by weight based on the total weight of the alcohols, and said catalyst comprising at least one metal selected from bismuth and gallium.

WO 2018/112777 A1 discloses a process for synthesizing at least one levulinate ester, said process comprising the reaction of furfuryl alcohol with at least one other alcohol in the presence of water and at least one catalyst, said furfuryl alcohol being present in a quantity of a least 5% by weight based on the total weight of the alcohols, and said catalyst comprising at least one triflate ligand and at least one metal selected from bismuth, gallium, aluminum, tin and iron.

WO 2018/112779 A1 discloses a process for synthesizing at least one levulinate ester, said process comprising the reaction of furfuryl alcohol polymer with at least one other alcohol in the presence of water and at least one catalyst, said catalyst comprising at least one metal selected from bismuth, gallium, aluminum, tin and iron, and said furfuryl alcohol polymer being obtainable by condensation of furfuryl alcohol at a temperature of at least 50° C.

The processes disclosed in WO 2018/112776 A1, WO 2018/112777 A1 and WO 2018/112779 A1 use specific catalysts which are less abundant and not as readily available as protic acid. Moreover, these specific catalysts are significantly more expensive than usual protic acids.

It is a primary object of the present invention to provide a process for forming alkyl ester of levulinic acid or one or more reaction products thereof (i.e. obtained by chemically converting said alkyl ester of levulinic acid), wherein acid-catalyzed homopolymerization of furfuryl alcohol is suppressed without the need to retain a part of the obtained alkyl ester of levulinic acid as solvent. It is a further object of the invention to omit the use of other catalysts than protic acid.

The primary object and other objects of the present invention can be accomplished by a process for forming alkyl ester of levulinic acid or one or more reaction products thereof (i.e. reaction products obtained by chemically converting said alkyl ester of levulinic acid),
said process comprising at least the following steps
(i) preparing or providing
   a first supply flux comprising furfuryl alcohol and aliphatic alcohol, and
   a second supply flux comprising at least one protic acid,
(ii) supplying the first supply flux and the second supply flux prepared or provided in step (i) to a pressurized continuous flow reactor system and contacting the first supply flux and the second supply flux in said pressurized continuous flow reactor system so that in the resulting reaction mixture alkyl ester of levulinic acid is formed by a protic acid-catalyzed reaction of furfuryl alcohol with said aliphatic alcohol,
   wherein the second supply flux supplies 2.6*10⁻² moles to 9*10⁻¹ moles, preferably 3*10⁻² moles to 5*10⁻¹ moles, more preferably 3*10⁻² moles to 3*10⁻¹ moles, most preferably 3*10⁻² moles to 7*10⁻² moles of protic acid per mole of furfuryl alcohol supplied by the first supply flux.

Surprisingly it has been found that by adjusting the molar ratio of protic acid supplied by the second supply flux and furfuryl alcohol supplied by the first supply flux in the above-defined range homopolymerization of furfuryl alcohol may be suppressed, and the selectivity of the formation of the target product levulinic acid is increased.

In step (i) of the process, a first supply flux comprising furfuryl alcohol and aliphatic alcohol and a second supply flux comprising at least one protic acid are prepared or provided.

It is understood by the skilled person that the first supply flux comprises substantially no protic acid, and the second supply flux comprises substantially no furfuryl alcohol, in order to avoid premature contact between furfuryl alcohol and protic acid which may result in acid-catalyzed homopolymerization of furfuryl alcohol.

As used herein, the term aliphatic alcohol denotes any individual aliphatic alcohol as well as mixtures of two or more different aliphatic alcohols. Said aliphatic alcohol in the first supply flux is preferably selected from the group consisting of methanol, ethanol, n-propanol, i-propanol, n-butanol and mixtures thereof.

Most preferred aliphatic alcohols for the process according to the invention are methanol and ethanol obtained from renewable sources like biomass, so that all starting materials (furfuryl alcohol as well as the aliphatic alcohol) used in the process are obtained from renewable sources.

In said first supply flux the concentration of furfuryl alcohol is preferably in the range of from 1 wt % to 40 wt %, more preferably of from 10 wt % to 25 wt %. In said first supply flux, the total concentration of aliphatic alcohol is preferably in the range of from 60 wt % to 99 wt %, more preferably of from 75 wt % to 90 wt %. Most preferably, in said first supply flux the concentration of furfuryl alcohol is in the range of from 1 wt % to 40 wt %, more preferably 10 wt % to 25 wt % and the total concentration of aliphatic alcohol is in the range of from 60 wt % to 99 wt %, more preferably 75 wt % to 90 wt %. Herein, the total concentration of aliphatic alcohol is the sum of the concentrations of all individual aliphatic alcohols in the first supply flux.

Said protic acid in the second supply flux is preferably not hydrochloric acid. More preferably, said protic acid in the second supply flux is selected from the group consisting of sulfuric acid, halogen sulfonic acids, aliphatic sulfonic acids, aromatic sulfonic acids and alkylaromatic sulfonic acids, preferably from the group consisting of sulfuric acid, methanesulfonic acid, para-toluenesulfonic acid, para-phenolsulfonic acid and naphthalenesulfonic acid. Most preferably, sulphuric acid is used as the protic acid catalyst.

Using hydrochloric acid as the catalyst is not preferred and not necessary. When the reaction mixture does not contain hydrochloric acid, the stability requirements to the reactor materials are less challenging. Thus, in particular when said protic acid supplied by the second supply flux is selected from the group consisting of sulfuric acid, halogen sulfonic acids, aliphatic sulfonic acids, aromatic sulfonic acids and alkyl-aromatic sulfonic acids, preferably from the group consisting of sulfuric acid, methanesulfonic acid, para-toluenesulfonic acid, para-phenolsulfonic acid and naphthalenesulfonic acid, said pressurized continuous flow reactor system may comprise a material selected from the group consisting of stainless steel and nickel alloys (especially those known under the trade name Hastelloy) which could not be used with hydrochloric acid as the catalyst.

Thus, in a preferred process according to the invention, said pressurized continuous flow reactor system comprises a material selected from the group consisting of stainless steel and nickel alloys, and said protic acid is selected from the group consisting of sulfuric acid, halogen sulfonic acids, aliphatic sulfonic acids, aromatic sulfonic acids and alkyl-aromatic sulfonic acids, preferably from the group consisting of sulfuric acid, methanesulfonic acid, para-toluenesulfonic acid, para-phenolsulfonic acid and naphthalenesulfonic acid, most preferably sulphuric acid.

Beyond protic acid, said second supply flux may further comprise aliphatic alcohol, wherein preferably said aliphatic alcohol present in said second supply flux is the same as in said first supply flux.

In said second supply flux, the total concentration of protic acid is preferably in the range of from 5 wt % to 100 wt %. In said second supply flux, the total concentration of aliphatic alcohol is preferably in the range of from 0 wt % to 95 wt %. Most preferably, in said second supply flux, the total concentration of protic acid is in the range of from 5 wt % to 100 wt %, and the total concentration of aliphatic alcohol is in the range of from 0 wt % to 95 wt %. Herein, the total concentration of protic acid is the sum of the concentrations of all individual protic acids in the second supply flux.

In step (ii) of the process, the first and second supply flux prepared or provided in step (i) (i.e. the first supply flux and the second supply flux as defined above) are supplied to a pressurized continuous flow reactor system, and the first and the second supply flux are contacted in said pressurized continuous flow reactor system so that in the resulting reaction mixture alkyl ester of levulinic acid is formed by a reaction of furfuryl alcohol with said aliphatic alcohol, said reaction being catalyzed by a protic acid.

As used herein, a pressurized continuous flow reactor system comprises a single reaction vessel configured for and used in pressurized continuous flow operation, or a combination of serially connected reaction vessels configured for and used in pressurized continuous flow operation. For further technical details of the pressurized continuous flow reactor system, please see the description of the apparatus according to the invention provided below.

It is not excluded that further supply fluxes are supplied to the pressurized continuous flow reactor system beyond said first supply flux and said second supply flux as defined above.

It is not excluded that one or more of the supply fluxes supplied to the pressurized continuous flow reactor system comprises minor amounts of alkyl ester of levulinic acid (for details see below).

The ratio of the total molar amount of aliphatic alcohol supplied to the pressurized continuous flow reactor system to the total molar amount of alkyl ester of levulinic acid supplied to the pressurized continuous flow reactor system is at least 3:1, preferably at least 10:1, more preferably at least 25:1, further preferably at least 100:1.

Herein, total molar amount of aliphatic alcohol is the sum of the molar amounts of all individual aliphatic alcohols supplied to the pressurized continuous flow reactor system, and total molar amount of alkyl esters of levulinic acid is the sum of the molar amounts of all individual alkyl esters of levulinic acid supplied to the pressurized continuous flow reactor system.

Nevertheless, in certain cases, it is preferred that no compound selected from the group consisting of levulinic acid and alkyl ester of levulinic acid is supplied to the pressurized continuous flow reactor system.

The term “pressurized continuous flow reactor system” means that the reaction mixture in the reactor system is maintained in liquid phase at the reaction temperature and a gas phase is not present, due to pressurizing by the vapor pressure of the liquid constituents of the reaction mixture. The pressure in the pressurized continuous flow reactor system may be in the range of from 0.1 to 10 MPa, preferably in the range of from 0.5 to 5 MPa.

In the pressurized continuous flow reactor system the furfuryl alcohol is allowed to react with the aliphatic alcohol in a reaction catalyzed by the protic acid, so that a product flux leaving the pressurized continuous flow reactor system results, said product flux having a ratio of the total molar amount of aliphatic alcohol to the total molar amount of alkyl ester of levulinic acid results which is lower than the ratio of the total molar amount of aliphatic alcohol supplied to the pressurized continuous flow reactor system to the total molar amount of alkyl ester of levulinic acid supplied to the pressurized continuous flow reactor system. Preferably, in said product flux leaving the pressurized continuous flow reactor system the ratio of the total molar amount of aliphatic alcohol to the total molar amount of alkyl ester of levulinic acid is preferably in the range of from 2:1 to 60:1, more preferably in the range of from 5:1 to 30:1.

The alkyl ester of levulinic acid formed by the process of the present invention is preferably selected from the group consisting of methyl levulinate, ethyl levulinate, n-propyl levulinate, i-propyl levulinate and n-butyl levulinate and mixtures thereof, more preferably from methyl levulinate and ethyl levulinate.

According to established research, it is assumed that the protic acid-catalyzed reaction of furfuryl alcohol with an aliphatic alcohol proceeds via fast formation of an intermediate (2-(alkoxymethyl) furan) which is stable in the presence of aliphatic alcohol and protic acid, and undergoes a slower consecutive reaction to form the target product alkyl ester of levulinic acid, but may also undergo undesired side reactions, especially decomposition and irreversible polymerization.

Fortunately, hydrochloric acid is not necessary to stabilize the intermediate 2-(alkoxymethyl)-furan, different from the preparation of levulinic acid, where the intermediate is a carbenium cation which needs stabilization by halogenide anions. In contrast, the intermediate 2-(alkoxymethyl) furan is stable in the absence of halogenide anions, so that hydrochloric acid may be omitted in the reaction mixture, and said protic acid catalyst may be selected from the group consisting of sulfuric acid, halogen sulfonic acids, aliphatic sulfonic acids, aromatic sulfonic acids and alkyl-aromatic sulfonic acids, preferably from the group consisting of sulfuric acid, methanesulfonic acid, para-toluenesulfonic acid, para-phenolsulfonic acid and naphthalenesulfonic acid, most preferably sulphuric acid.

An important measure to avoid polymerization of furfuryl alcohol is fast dilution of the furfuryl alcohol in the reaction mixture. This is achieved by common means like stirring, injection, and back-mixing of the reaction mixture, and by controlling the concentration of furfuryl alcohol and protic acid in the reaction mixture by suitable adjustment of the supply flux of furfuryl alcohol and of the supply flux of protic acid.

As the conversion of furfuryl alcohol to the intermediate proceeds complete and fast, it is of utmost importance to provide reaction conditions which accelerate and promote conversion of the intermediate to the target product in order to avoid polymerization of the intermediate.

It has been found that by adjusting one or more parameters from the group consisting of
the molar ratio of protic acid supplied by the second supply flux relative to furfuryl alcohol supplied by the first supply flux
the concentration of furfuryl alcohol in the reaction mixture resulting from contacting the first and second supply flux in the pressurized continuous flow reactor system the concentration of protic acid in the reaction mixture resulting from contacting the first and second supply flux in the pressurized continuous flow reactor system
the temperature in said pressurized continuous flow reactor system
the residence time in said pressurized continuous flow reactor system
   a selectivity of the formation of alkyl esters of levulinic acid of 70% or more, preferably 80% or more may be achieved.

Thus, it is preferred that in the process according to the invention one or more parameters, preferably all parameters from the group consisting of
the molar ratio of protic acid supplied by the second supply flux relative to furfuryl alcohol supplied by the first supply flux
the concentration of furfuryl alcohol in the reaction mixture resulting from contacting the first and second supply flux in the pressurized continuous flow reactor system
the concentration of protic acid in the reaction mixture resulting from contacting the first and second supply flux in the pressurized continuous flow reactor system
the temperature in said pressurized continuous flow reactor system
the residence time in said pressurized continuous flow reactor system
   are adjusted so that the selectivity of the formation of alkyl esters of levulinic acid is 70% or more, preferably 80% or more.

Preferably, the concentration of furfuryl alcohol in the reaction mixture resulting from contacting the first and second supply flux in the pressurized continuous flow reactor system is less than 15 wt %, further preferably less than 10 wt %, more preferably less than 5 wt %.

When the concentration of furfuryl alcohol is too high, homopolymerization of furfuryl alcohol may be promoted, resulting in a loss of yield of the target product alkyl ester of levulinic acid, and formation of polymeric sediments which may damage the process equipment. When the concentration of furfuryl alcohol is too low, the space-time yield may be too low.

Preferably, the concentration of protic acid in the reaction mixture resulting from contacting the first and second supply flux in the pressurized continuous flow reactor system is in the range of from 0.1 wt % to 1.5 wt %. When the concentration of protic acid is too high, decomposition of the target product alkyl ester of levulinic acid may be promoted. When the concentration of protic acid is too low, the consecutive reaction of the intermediate 2-(alkoxymethyl) furan to the target product alkyl ester of levulinic acid may become too slow, so that side reactions are promoted, and the space-time yield may be too low.

Preferably, the temperature in the pressurized continuous flow reactor system is in the range of from 105° C. to 160° C., more preferably 115° C. to 145° C. When the temperature is too high, side reactions and decomposition of the target product may be promoted, resulting in a loss of yield of the target product alkyl ester of levulinic acid. When the temperature is too low, the consecutive reaction of the intermediate 2-(alkoxymethyl) furan to the target product alkyl ester of levulinic acid may become too slow, so that side reactions are promoted, and the space-time yield may be too low.

Preferably, the residence time in the pressurized continuous flow reactor system is in the range of from 0.25 to 5.0 hours, more preferably 0.5 to 3.0 hours. When the residence time is too high, side reactions may be promoted, resulting in a loss of yield of the target product alkyl ester of levulinic acid. When the residence time is too low, the degree of conversion of furfuryl alcohol may be too low, and the consecutive reaction of the intermediate 2-(alkoxymethyl) furan to the target product alkyl ester of levulinic acid may be not complete.

Most preferably,
the concentration of furfuryl alcohol in the reaction mixture resulting from contacting the first and second supply flux in the pressurized continuous flow reactor system is less than 15 wt %, preferably less than 10 wt %, more preferably less than 5 wt %
   and
the concentration of protic acid in the reaction mixture resulting from contacting the first and second supply flux in the pressurized continuous flow reactor system is in the range of from 0.1 wt % to 1.5 wt %
   and
the temperature in the pressurized continuous flow reactor system is in the range of from 105° C. to 160° C., preferably 115° C. to 145° C.,
   and
the residence time in the pressurized continuous flow reactor system is in the range of from 0.25 to 5.0 hours, preferably 0.5 to 3.0 hours.

Preferably, the reaction mixture does not comprise a catalyst comprising least one metal selected from bismuth, gallium, aluminum, tin and iron.

Preferably, the reaction mixture does not comprise a catalyst comprising at least one triflate ligand (trifluoromethanesulfonate CF₃SO₃⁻) and at least one metal selected from bismuth, gallium, aluminum, tin and iron.

In certain cases, it is preferred that no compound selected from the group consisting of compounds of Bi, Ga, Al, Sn and Fe is present in the reaction mixture. Most preferably, no Lewis acid is present in the reaction mixture.

In preferred processes according to the invention, non-reacted aliphatic alcohol is separated from the product flux leaving the pressurized continuous flow reactor system and said non-reacted aliphatic alcohol is recycled to said pressurized continuous flow reactor system wherein alkyl ester of levulinic acid is formed by a protic acid-catalyzed reaction of furfuryl alcohol with said aliphatic alcohol. Separation of non-reacted alcohol may be carried out by means of distillation.

Recycling of non-reacted aliphatic alcohol separated from the product flux to the pressurized continuous flow reactor may be achieved by feeding the recycled aliphatic alcohol into one or both of the first supply flux and the second supply flux, and/or by supplying the recycled aliphatic alcohol as a further supply flux directly to said pressurized continuous flow reactor system.

Said recycled aliphatic alcohol may comprise minor contaminations by the target product (alkyl ester of levulinic acid). Preferably, in the process according to the invention alkyl ester of levulinic acid admixed to aliphatic alcohol recycled from the product flux leaving the pressurized continuous flow reactor system is the only source of alkyl ester of levulinic acid supplied to the pressurized continuous flow reactor system.

In certain cases, a process according to the invention further comprises the step of
(iii) chemically converting alkyl ester of levulinic acid formed in step (ii) into one or more reaction products, wherein chemically converting said alkyl ester of levulinic acid into one or more reaction products comprises elimination of aliphatic alcohol.

Step (iii) of the process according to the invention may comprise one or more sub-steps to obtain the desired reaction product. In at least one of said sub-steps, aliphatic alcohol is eliminated. In other sub-steps, hydrogenation reactions or dehydration reactions may be carried out.

Said one or more reaction products obtained in step (iii) may be selected from the group consisting of levulinic acid, ketals of alkyl esters of levulinic acid, 1,4-pentanediol, gamma-valerolactone, alpha-angelicalactone, beta-angelicalactone, 2-methyl-tetrahydrofuran, 4-hydroxy-pentanal, 5-methyl-2-hydroxy-tetrahydrofuran, pent-4-en-1-ol, 1-pentanol, 4-pentanol, pent-4-en-al, pentanal (valeraldehyde), pentanoic acid (valeric acid), alkyl esters of pentanoic acid, 4-hydroxyvaleric acid and alkyl esters of 4-hydroxyvaleric acid.

Aliphatic alcohol eliminated in step (iii) may be recycled to said pressurized continuous flow reactor system wherein alkyl ester of levulinic acid is formed by a protic acid-catalyzed reaction of furfuryl alcohol with said aliphatic alcohol.

Recycling of aliphatic alcohol eliminated in step (iii) to the pressurized continuous flow reactor system may be achieved by feeding the recycled aliphatic alcohol into one or both of the first supply flux and the second supply flux, and/or by supplying the recycled aliphatic alcohol as a further supply flux directly to said pressurized continuous flow reactor system.

Said recycled aliphatic alcohol may comprise minor contaminations by the target product (alkyl ester of levulinic acid). Preferably, in the process according to the invention alkyl ester of levulinic acid admixed to aliphatic alcohol recycled from the product flux leaving the pressurized continuous flow reactor system and/or recycled from step (iii) is the only source of alkyl ester of levulinic acid supplied to the pressurized continuous flow reactor system.

A specifically preferred process according to the invention is a process for forming an alkyl ester of levulinic acid selected from the group consisting of methyl levulinate, ethyl levulinate, n-propyl levulinate, i-propyl levulinate and n-butyl levulinate and mixtures thereof, preferably methyl levulinate or ethyl levulinate, or one or more reaction products thereof comprises the steps of
(i) preparing or providing
   a first supply flux comprising furfuryl alcohol and aliphatic alcohol, wherein said aliphatic alcohol is selected from the group consisting of methanol, ethanol, n-propanol, i-propanol, n-butanol and mixtures thereof, preferably from methanol and ethanol,
   and
   a second supply flux comprising at least one protic acid, wherein said protic acid is selected from the group consisting of sulfuric acid, halogen sulfonic acids, aliphatic sulfonic acids, aromatic sulfonic acids and alkyl-aromatic sulfonic acids, preferably sulphuric acid,
(ii) supplying the first supply flux and the second supply flux prepared or provided in step (i) to a pressurized continuous flow reactor system and contacting the first supply flux and the second supply flux in said pressurized continuous flow reactor system so that in the resulting reaction mixture alkyl ester of levulinic acid is formed by a protic acid-catalyzed reaction of furfuryl alcohol with said aliphatic alcohol,
   wherein the second supply flux supplies 3*10⁻² moles to 5*10⁻¹ moles, preferably 3*10⁻² moles to 3*10⁻¹ moles, more preferably 3*10⁻² moles to 7*10⁻² moles of protic acid per mole of furfuryl alcohol supplied by the first supply flux,
   wherein
      the concentration of furfuryl alcohol in the reaction mixture resulting from contacting the first and second supply flux in the pressurized continuous flow reactor system is less than 15 wt %, preferably less than 10 wt %, more preferably less than 5 wt %
      the concentration of protic acid in the reaction mixture resulting from contacting the first and second supply flux in the pressurized continuous flow reactor system is in the range of from 0.1 wt % to 1.5 wt %
      the temperature in the pressurized continuous flow reactor system is in the range of from 105° C. to 160° C., preferably 115° C. to 145° C.
      and the residence time in the pressurized continuous flow reactor system is in the range of from 0.25 to 5.0 hours, preferably 0.5 to 3.0 hours
   wherein no compound selected from the group consisting of compounds of Bi, Ga, Al, Sn and Fe, is present in the reaction mixture.

In said specifically preferred process non-reacted aliphatic alcohol may be separated from the product flux leaving the pressurized continuous flow reactor system and said non-reacted aliphatic alcohol is recycled to said pressurized continuous flow reactor system wherein alkyl ester of levulinic acid is formed by a protic acid-catalyzed reaction of furfuryl alcohol with said aliphatic alcohol.

Said specifically preferred process may further comprise the step of
(iii) chemically converting alkyl ester of levulinic acid formed in step (ii) into one or more reaction products, wherein chemically converting said alkyl ester of levulinic acid into one or more reaction products comprises elimination of aliphatic alcohol,
   wherein said one or more reaction products are preferably selected from the group consisting of levulinic acid, 1,4-pentanediol, gamma-valerolactone, alpha-angelicalactone, beta-angelicalactone and 2-methyl-tetrahydrofuran,
   wherein optionally aliphatic alcohol eliminated in step (iii) is recycled to said pressurized continuous flow reactor system wherein alkyl ester of levulinic acid is formed by a protic acid-catalyzed reaction of furfuryl alcohol with said aliphatic alcohol.

A further aspect of the present invention is the use of a pressurized continuous flow reactor system for forming alkyl ester of levulinic acid by a protic acid-catalyzed reaction of furfuryl alcohol with aliphatic alcohol by a process according to the invention as described above, preferably by one of the preferred processes according the invention as described above.

Using hydrochloric acid as the catalyst is not preferred and not necessary. When the reaction mixture does not contain hydrochloric acid, the stability requirements to the reactor system materials are less challenging. Thus, in particular when said protic acid is selected from the group consisting of sulfuric acid, halogen sulfonic acids, aliphatic sulfonic acids, aromatic sulfonic acids and alkyl-aromatic sulfonic acids, preferably from the group consisting of sulfuric acid, methanesulfonic acid, para-toluenesulfonic acid, para-phenolsulfonic acid, naphthalenesulfonic acid, said pressurized continuous flow reactor system may comprise a material selected from the group consisting of stainless steel and nickel alloys (especially those known under the trade name Hastelloy) which could not be used with hydrochloric acid as the catalyst.

A further aspect of the invention is an apparatus for carrying out the process according to the invention as defined above. Said apparatus comprises
a first supply line and a second supply line
a pressurized continuous flow reactor system in fluid communication with said first supply line and said second supply line
an exit line in fluid communication with said pressurized continuous flow reactor system
means for adjusting the molar ratio of protic acid supplied by the second supply flux relative to furfuryl alcohol supplied by the first supply flux, so that the second supply flux supplies 2.6*10⁻² moles to 9*10⁻¹ moles, preferably 3*10⁻² moles to 5*10⁻¹ moles, more preferably 3*10⁻² moles to 3*10⁻¹ moles, most preferably 3*10⁻² moles to 7*10⁻² moles of protic acid per mole of furfuryl alcohol supplied by the first supply flux,
   wherein
said first supply line is configured to supply a first supply flux comprising furfuryl alcohol and aliphatic alcohol
said second supply line is configured to supply a second supply flux comprising at least one protic acid,
said pressurized continuous flow reactor system is configured for contacting the first supply flux supplied by the first supply line and the second supply flux supplied by the second supply line so that in the resulting reaction mixture alkyl ester of levulinic acid is formed by a protic acid-catalyzed reaction of furfuryl alcohol with said aliphatic alcohol,
said exit line is configured to allow a product flux comprising alkyl ester of levulinic acid formed in the pressurized continuous flow reactor system and non-reacted aliphatic alcohol to leave the pressurized continuous flow reactor system,
   wherein said pressurized continuous flow reactor system comprises
means for contacting the first supply flux supplied by the first supply line and the second supply flux supplied by the second supply line
means for pressurizing the pressurized continuous flow reactor system.

The pressurized continuous flow reactor system is in fluid communication with a first supply line and a second supply line. Said first supply line is configured to supply a first supply flux comprising furfuryl alcohol and aliphatic alcohol, and said second supply line is configured to supply a second supply flux comprising at least one protic acid. The first supply line and the second supply line are operated independently from each other.

The pressurized continuous flow reactor system is configured for contacting the first supply flux supplied by the first supply line and the second supply flux supplied by the second supply line so that in the resulting reaction mixture alkyl ester of levulinic acid is formed by a protic acid-catalyzed reaction of furfuryl alcohol with said aliphatic alcohol.

The pressurized continuous flow reactor system may comprise a single reaction vessel configured for and used in pressurized continuous flow operation, or a combination of serially connected reaction vessels configured for and used in pressurized continuous flow operation. Said continuous flow reactor system may be selected from the group consisting of a continuous stirred tank reactor, a flow tube, a tubular reactor, a jet loop reactor, a cascade of continuous stirred tank reactors, and combinations thereof.

In the “pressurized continuous flow reactor system” the reaction is maintained in liquid phase at the reaction temperature and a gas phase is not present, due to pressurizing by the vapor pressure of the liquid constituents of the reaction mixture. Pressurizing may be achieved by means of a pressure retention valve. The pressure in the pressurized continuous flow reactor system may be in the range of from 0.1 to 10 MPa, preferably in the range of from 0.5 to 5 MPa.

Most preferably, the pressurized continuous flow reactor system comprises a back-mixed reactor optionally followed by a residence time section which may be in the form of a tubular reactor (flow tube) or a cascade of back-mixed reactors. It has been found that such setup allows for high dilution and fast conversion of furfuryl alcohol in the back-mixed reactor, followed by complete conversion of the intermediate 2-(alkoxymethyl) furan in the residence time section.

The pressurized continuous flow reactor system comprises means for contacting the first supply flux supplied by the first supply line and the second supply flux supplied by the second supply line.

The pressurized continuous flow reactor system may be in fluid communication with one or more further supply lines.

In the process according to the invention, which is carried out in the above-defined apparatus, use of hydrochloric acid as the catalyst is not preferred and not necessary. When the reaction mixture does not contain hydrochloric acid, the stability requirements to the reactor materials are less challenging. Thus, in particular when said protic acid is selected from the group consisting of sulfuric acid, halogen sulfonic acids, aliphatic sulfonic acids, aromatic sulfonic acids and alkyl-aromatic sulfonic acids, preferably from the group consisting of sulfuric acid, methanesulfonic acid, para-toluenesulfonic acid, para-phenolsulfonic acid, naphthalenesulfonic acid, said pressurized continuous flow reactor system may comprise a material selected from the group consisting of stainless steel and nickel alloys (especially those known under the trade name Hastelloy) which could not be used with hydrochloric acid as the catalyst.

In the apparatus according to the invention, an exit line is in fluid communication with said pressurized continuous flow reactor system. Said exit line is configured to allow a product flux comprising alkyl ester of levulinic acid formed in the pressurized continuous flow reactor system and non-reacted aliphatic alcohol to leave the pressurized continuous flow reactor system.

Preferably, the apparatus comprises means for adjusting one or more parameters from the group consisting of
the molar ratio of protic acid supplied by the second supply flux relative to furfuryl alcohol supplied by the first supply flux
the concentration of furfuryl alcohol in the reaction mixture resulting from contacting the first and second supply flux in the pressurized continuous flow reactor system
the concentration of protic acid in the reaction mixture resulting from contacting the first and second supply flux in the pressurized continuous flow reactor system
the temperature in said pressurized continuous flow reactor system
the residence time in said pressurized continuous flow reactor system.

Means for adjusting the above-mention parameters are known to the skilled person. For instance, a thermostat may be used for adjusting the temperature. For instance, pumps may be used for adjusting the residence time. For instance, pumps may be used for adjusting the molar ratio of protic acid supplied by the second supply flux relative to furfuryl alcohol supplied by the first supply flux. For instance, pumps may be used for adjusting the concentration of furfuryl alcohol in the reaction mixture resulting from contacting the first and second supply flux in the pressurized continuous flow reactor system.

Preferably the above-mentioned parameters are adjusted so that they fall in the above-defined preferred ranges.

In certain cases, said pressurized continuous flow reactor system comprises one or more of
a stirrer
means for flow-breaking
a nozzle
a deflector plate
means for static mixing
   which serve to provide for fast dilution of furfuryl alcohol and for substantially complete mixing of the reactants.

In certain cases, the apparatus further comprises
means for separating non-reacted aliphatic alcohol from the product flux leaving the pressurized continuous flow reactor system via the exit line, and
means for recycling said non-reacted aliphatic alcohol separated from the product flux to said pressurized continuous flow reactor system configured for forming a reaction mixture wherein alkyl ester of levulinic acid is formed by a protic acid-catalyzed reaction of furfuryl alcohol with said aliphatic alcohol.

Separation of non-reacted alcohol may be carried out by means of distillation.

Recycling of non-reacted aliphatic alcohol separated from the product flux to the pressurized continuous flow reactor system may be achieved by feeding the recycled aliphatic alcohol into one or both of the first supply line and the second supply line, and/or by supplying the recycled aliphatic alcohol via a further supply line directly to the pressurized continuous flow reactor system.

The apparatus may comprise a second reactor system in fluid communication with said exit line of said pressurized continuous flow reactor system, or in fluid communication with an exit line of the means for separating non-reacted aliphatic alcohol from the product flux leaving the pressurized continuous flow reactor system via the exit line. Said second reactor system is configured for chemically converting alkyl ester of levulinic acid formed in the pressurized continuous flow reactor system into one or more reaction products, wherein chemically converting alkyl ester of levulinic acid into one or more reaction products comprises elimination of aliphatic alcohol. Said second reactor system may be a continuous flow reactor system, a semi batch reactor system or a batch reactor system.

The apparatus comprising a second reactor system as defined above may further comprise
means for separating eliminated aliphatic alcohol from the reactor system resp. from the product flux leaving the second reactor system via the exit line, and
means for recycling aliphatic alcohol eliminated in the second reactor system to the pressurized continuous flow reactor system configured for contacting the first supply flux and the second supply flux so that in the resulting reaction mixture alkyl ester of levulinic acid is formed by a protic acid-catalyzed reaction of furfuryl alcohol with said aliphatic alcohol.

Usually, the second reactor system resp. the means for separating eliminated aliphatic alcohol from the product flux leaving the second reactor system via the exit line comprises an exit line configured to allow a product flux comprising one or more reaction products obtained by chemically converting alkyl ester of levulinic acid formed in the pressurized continuous flow reactor system to leave the second reactor system resp. to leave the means for separating eliminated aliphatic alcohol from the product flux leaving the second reactor system.

Recycling of aliphatic alcohol eliminated in the second reactor system to the pressurized continuous flow reactor system may be achieved by feeding the recycled aliphatic alcohol into one or both of the first supply line and the second supply line, and/or by supplying the recycled aliphatic alcohol via a further supply line directly into said pressurized continuous flow reactor system.

In a specifically preferred apparatus, said second reactor system is configured for gas phase conversion of ethyl levulinate with hydrogen to gammavalerolacton (GVL) under elimination of ethanol.

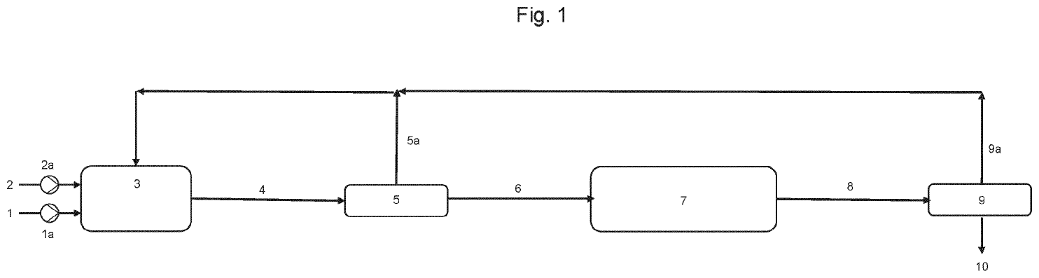

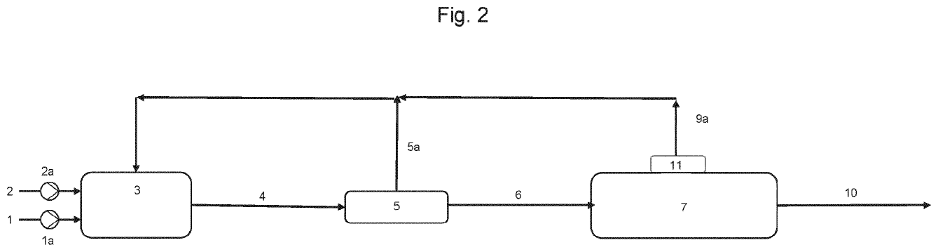

An exemplary apparatus according to the invention is shown in FIG. 1. Another exemplary apparatus according to the invention is shown in FIG. 2. Said apparatus comprises
a first supply line 1 and a second supply line 2
a pressurized continuous flow reactor system 3 in fluid communication with said first supply line 1 and said second supply line 2
an exit line 4 in fluid communication with said pressurized continuous flow reactor system 3
means 1a, 2a for adjusting the molar ratio of protic acid supplied by the second supply flux relative to furfuryl alcohol supplied by the first supply flux, so that the second supply flux supplies 2.6*10⁻² moles to 9*10⁻¹ moles, preferably 3*10⁻² moles to 5*10⁻¹ moles, more preferably 3*10⁻² moles to 3*10⁻¹ moles, most preferably 3*10⁻² moles to 7*10⁻² moles of protic acid per mole of furfuryl alcohol supplied by the first supply flux
   wherein
said first supply line 1 is configured to supply a first supply flux comprising furfuryl alcohol and aliphatic alcohol
said second supply line 2 is configured to supply a second supply flux comprising at least one protic acid,
said pressurized continuous flow reactor system 3 is configured for contacting the first supply flux supplied by the first supply line 1 and the second supply flux supplied by the second supply line 2 so that in the resulting reaction mixture alkyl ester of levulinic acid is formed by a protic acid-catalyzed reaction of furfuryl alcohol with said aliphatic alcohol,
said exit line 4 is configured to allow a product flux comprising alkyl ester of levulinic acid formed in the pressurized continuous flow reactor system 3 and non-reacted aliphatic alcohol to leave the pressurized continuous flow reactor system 3,
   wherein said pressurized continuous flow reactor system 3 comprises
means for contacting the first supply flux supplied by the first supply line 1 and the second supply flux supplied by the second supply line 2 (not shown in FIGS. 1 and 2)
means for pressurizing the pressurized continuous flow reactor system (not shown in FIGS. 1 and 2).

The pressurized continuous flow reactor system 3 may be in fluid communication with one or more further supply lines 5a, 9a.

In certain cases, the apparatus further comprises
means 5 for separating non-reacted aliphatic alcohol from the product flux leaving the pressurized continuous flow reactor system 3 via the exit line 4, and
means 5a for recycling said non-reacted aliphatic alcohol separated from the product flux to said pressurized continuous flow reactor system 3 configured for forming a reaction mixture wherein alkyl ester of levulinic acid is formed by a protic acid-catalyzed reaction of furfuryl alcohol with said aliphatic alcohol.

Recycling of non-reacted aliphatic alcohol separated from the product flux to the pressurized continuous flow reactor system 3 may be achieved by feeding the recycled aliphatic alcohol into one or both of the first supply line 1 and the second supply line 2 (not shown in FIGS. 1 and 2), and/or by supplying the recycled aliphatic alcohol via a further supply line 5a directly to the pressurized continuous flow reactor system 3.

The apparatus may comprise a second reactor system 7 in fluid communication with said exit line 4 of said pressurized continuous flow reactor system, or with an exit line 6 of the means 5 for separating non-reacted aliphatic alcohol from the product flux leaving the pressurized continuous flow reactor system 3 via the exit line 4. Said second reactor system 7 is configured for chemically converting alkyl ester of levulinic acid formed in the pressurized continuous flow reactor system 3 into one or more reaction products, wherein chemically converting alkyl ester of levulinic acid into one or more reaction products comprises elimination of aliphatic alcohol.

The apparatus comprising a second reactor system 7 as defined above may further comprise
means 9 for separating eliminated aliphatic alcohol from the product flux leaving the second reactor system 7 via the exit line 8 (cf. FIG. 1), resp. means 11 for separating eliminated aliphatic alcohol from the reactor system 7 (cf. FIG. 2)
   and
means 9a for recycling aliphatic alcohol eliminated in the second reactor system 7 to the pressurized continuous flow reactor system 3 configured for contacting the first supply flux and the second supply flux so that in the resulting reaction mixture alkyl ester of levulinic acid is formed by a protic acid-catalyzed reaction of furfuryl alcohol with said aliphatic alcohol.

Usually, the second reactor system 7 (cf. FIG. 2) resp. the means 9 (cf. FIG. 1) for separating eliminated aliphatic alcohol from the product flux leaving the second reactor system 7 via the exit line 8 comprises an exit line 10 configured to allow a product flux comprising one or more reaction products obtained by chemically converting said alkyl ester of levulinic acid formed in the pressurized continuous flow reactor system 3 to leave the second reactor system 7 resp. the means 9 for separating eliminated aliphatic alcohol from the product flux leaving the second reactor system 7.

Recycling of aliphatic alcohol eliminated in the second reactor system 7 to the pressurized continuous flow reactor system 3 may be achieved by feeding the recycled aliphatic alcohol into one or both of the first supply line 1 and the second supply line 2 (not shown in FIGS. 1 and 2), and/or by supplying the recycled aliphatic alcohol via a further supply line 9a, 5a directly into said pressurized continuous flow reactor system 3.

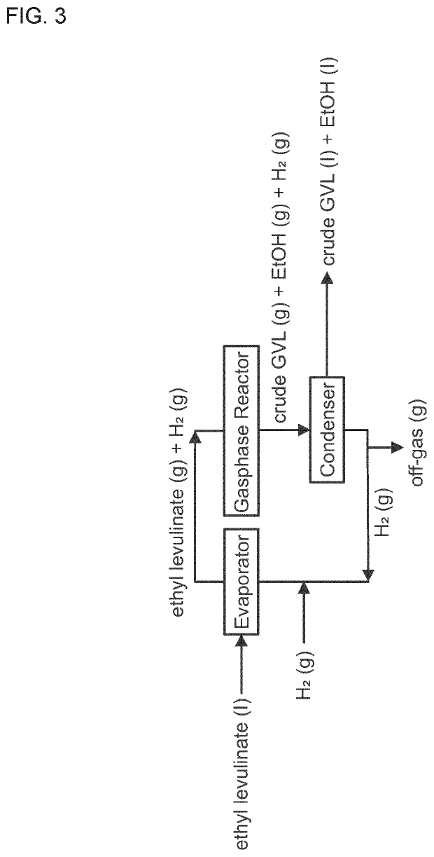

A second reactor system configured for gas phase conversion of ethyl levulinate with hydrogen to gamma-valerolacton (GVL) under elimination of ethanol is shown in FIG. 3.

Liquid ethyl levulinate is supplied to an evaporator where it is transferred into the gas phase. Together with the evaporated ethyl levulinate, hydrogen gas is supplied to a gas phase reactor, wherein ethyl levulinate is hydrogenated to gamma-valerolacton under elimination of ethanol. The gaseous reaction mixture leaving the gas phase reactor is supplied to a condenser for liquefying and separating crude gamma-valerolacton and ethanol. Non-used hydrogen gas is recycled, while off-gas is separated.

### EXAMPLES

The following examples are meant to further explain and illustrate the present invention without limiting its scope.

### 1. Reactor System and General Procedure

For the basic kinetic investigation of the formation of alkyl ester of levulinic acid by protic acid-catalyzed reaction of furfuryl alcohol with an aliphatic alcohol, a pressurized (by the vapor pressure of the used aliphatic alcohol) stop-flow reactor system was used, which is described below. The stop-flow reactor system is advantageous for such basic investigations, because it constitutes a combination of continuous operation and discontinuous operation. The continuously operating part of the experiment allows to start the reaction at defined conditions, i.e. temperature, pressure, residence time and concentration of reactants. Effects of mixing the reactants and heating the mixture to a certain temperature at a certain pressure could be suppressed. Furthermore, it turned out that the conversion of furfuryl alcohol to the intermediate is relatively fast, whereas completion of the formation of the alkyl ester of levulinic acid could take considerably longer. Thus, the discontinuously operating (batch mode) part of the experiment served to monitor consumption of reaction intermediates, formation of the target product and determination of the selectivity of the reaction.

In the stop-flow reactor system, the reaction takes place in a 270 mL nickel alloy autoclave. The autoclave is in fluid connection with two independently operated supply lines under automated control and equipped with electronic balances to obtain a mass balance of the reaction. The first supply line supplies a first supply flux comprising furfuryl alcohol and aliphatic alcohol, the second line supplies a second supply flux comprising sulfuric protic acid and an aliphatic alcohol which is the same as in the first supply flux. The supply rates are adjusted by two pumps independently, so that the average residence time in the autoclave and the concentration of sulfuric acid in the autoclave can be adjusted. The autoclave is equipped with a thermostat for temperature control, a stirrer and flow breaker. A high stirring speed of 1000 rpm is used to guarantee substantially complete mixing and fast dilution of the added furfuryl alcohol.

The autoclave is equipped with a sampling outlet and an exit to a needle valve, which is controlled by pressure (set to the vapor pressure of the used aliphatic alcohol at the target temperature).

In each experiment, a first supply flux comprising furfuryl alcohol (FFA) admixed to an aliphatic alcohol selected from methanol, ethanol and n-butanol was pumped through the reactor system and an average residence time of 0.5 to 1.5 h in the autoclave was adjusted in continuous mode. The pump rates were adjusted to achieve the target residence time in the autoclave, and the desired concentration of furfuryl alcohol (cf. column “FFA [wt %]” in tables 1-10 below) and the desired concentration of sulfuric acid (cf. column “H₂SO₄ [wt %]” in tables 1-10 below) in the reaction mixture. The temperature was increased to the target temperature and dosing of the second supply flux comprising sulfuric acid in the same aliphatic alcohol which is present in the first supply flux was started, and the first supply flux and the second supply flux were pumped continuously through the autoclave. The reaction was run at target temperature under stirring at 1000 rpm to achieve substantially complete mixing. After at least 5 average residence times (achieving steady state and a >99% exchange of the reaction mixture), the supply lines and the exit line of the autoclave were closed, and the reaction was continued in batch mode. Samples were taken at scheduled times and analyzed by gas chromatography (GC) using 1-methoxy-2-(2-methoxyethoxy) ethane (also known as diglyme or DEGDME) as internal standard. The obtained wt % data were then used to calculate the conversion of furfuryl alcohol (cf. column “conv. of FFA [%]” in tables 1-10 below) and the selectivity with respect to the formation of the intermediate 2-(alkoxymethyl) furan and of the target product (alkyl ester of levulinic acid) (cf. columns “Selectivity . . . [%]” in tables 1-13 below).

The conversion (in %), yield (in %) and selectivity (in %) for each product were determined as defined by the following equations. Conversion, selectivity and yield are all calculated on a molar basis.

$$conversion \% = \frac{moles of initial reactant - moles of residual reactant}{moles of initial reactant} * 100$$

$$yield \% = \frac{moles of obtained product}{moles of theoretical product} * 100$$

$$selectivity \% = \frac{yield}{conversion} * 100$$

### 2. Formation of Ethyl Ester of Levulinic Acid (Ethyl Levulinate)

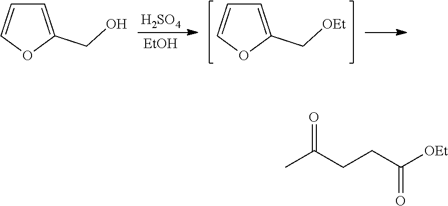

The formation of ethyl levulinate was investigated using the stop-flow reactor and the procedure described above.

At a temperature of 105° C. and using a reaction mixture comprising 0.5 wt % sulfuric acid and 10% furfuryl alcohol in ethanol, substantially full conversion of furfuryl alcohol and also of the intermediate were obtained after 2.5 h (table 1, experiment 1). The selectivity of the formation of ethyl levulinate reaches 68.5%. When decreasing the concentration of sulfuric acid in the reaction mixture to 0.1 or 0.05 wt % (table 1, experiments 2 and 3), full conversion of furfuryl alcohol was obtained, too, but the reaction to the target product was much slower. When the selectivity for the intermediate 2-(ethoxymethyl) furan and the selectivity for ethyl levulinate are summed up, the overall results were inferior to the experiments with 0.5 wt % sulfuric acid. As the conversion of furfuryl alcohol is complete and fast under all tested reaction conditions, it seems necessary to reach a fast conversion of the intermediate to avoid polymerization. When the temperature was increased to 115° C., the same trends of the selectivity were observed when reducing the concentration of sulfuric acid in the reaction mixture from 0.5 wt % (74.7% ethyl levulinate selectivity) to 0.1 or 0.05 wt % (57.6% or 52.8% ethyl levulinate selectivity) (table 1, experiments 4 to 8).

TABLE 1
Influence of variation of the concentration of sulfuric acid and/or of FFA in the
reaction mixture at 105° C. and 115° C. on the formation of ethyl
levulinate. Residence time in continuous mode: 0.5 hours

| No. of | | | | time of | Conver- | Selectivity | Selectivity |
|---|---|---|---|---|---|---|---|
| experi- | | H₂SO₄ | FFA | sample | sion of | 2-(ethoxy- | ethyl levulinate |
| ment | T [° C.] | [wt %] | [wt %] | taking [h] | FFA [%] | methyl) furan [%] | [%] |
| 1 | 105 | 0.5 | 10 | 0 | 98.5 | 9.4 | 36.8 |
| | | | | 0.5 | 100 | 9.5 | 53.0 |
| | | | | 2.5 | 100 | 0.1 | 68.5 |
| 2 | 105 | 0.1 | 10 | 0 | 87.6 | 19.9 | 16.9 |
| | | | | 1.0 | 100 | 15.9 | 28.7 |
| | | | | 2.5 | 100 | 6.0 | 42.9 |
| 3 | 105 | 0.05 | 10 | 0 | 74.5 | 18.0 | 8.2 |
| | | | | 2.5 | 100 | 14.2 | 26.9 |
| 4 | 115 | 0.5 | 10 | 0 | 99.2 | 8.5 | 42.1 |
| | | | | 0.17 | 100 | 5.2 | 58.1 |
| | | | | 1.0 | 100 | 0 | 67.4 |
| | | | | 2.5 | 100 | 0 | 69.4 |
| 5 | 115 | 0.5 | 5 | 0 | 99.9 | 9.4 | 45.4 |
| | | | | 1.0 | 100 | 0 | 71.9 |
| | | | | 2.5 | 100 | 0 | 73.4 |
| 6 | 115 | 0.5 | 5 | 5.0 | 100 | 0 | 74.7 |
| 7 | 115 | 0.1 | 10 | 0 | 95.4 | 14.2 | 33.1 |
| | | | | 0.5 | 100 | 11.7 | 40.7 |
| | | | | 2.5 | 100 | 0.7 | 57.6 |
| 8 | 115 | 0.05 | 10 | 0 | 87.1 | 21.6 | 18.3 |
| | | | | 2.5 | 100 | 4.4 | 52.8 |

As fast conversion of furfuryl alcohol and of the intermediate are necessary, the temperature was further increased (table 2).

TABLE 2
Influence of increased temperature on the formation of ethyl levulinate. Residence
time in continuous mode: 0.5 hours

| No. of | | | | time of | | Selectivity | Selectivity |
|---|---|---|---|---|---|---|---|
| experi- | | H₂SO₄ | FFA | sample | Conversion | 2-(ethoxy- | ethyl |
| ment | T [° C.] | [wt %] | [wt %] | taking [h] | of FFA [%] | methyl) furan [%] | levulinate [%] |
| 9 | 125 | 0.5 | 10 | 0 | 99.6 | 6.4 | 62.6 |
| | | | | 0.75 | 100 | 0 | 75.2 |
| | | | | 2.5 | 100 | 0 | 77.9 |
| 10 | 125 | 0.5 | 5 | 0 | 100 | 6.1 | 66.3 |
| | | | | 0.5 | 100 | 0 | 77.9 |
| | | | | 2.5 | 100 | 0 | 82.3 |
| 11 | 135 | 0.5 | 10 | 0 | 100 | 3.6 | 71.9 |
| | | | | 0.25 | 100 | 0 | 84.8 |
| | | | | 2.5 | 100 | 0 | 88.9 |
| 12 | 135 | 0.5 | 5 | 0 | 100 | 2.2 | 77.4 |
| | | | | 0.25 | 100 | 0 | 88.3 |
| | | | | 2.5 | 100 | 0 | 91.4 |
| 13 | 135 | 0.75 | 5 | 0 | 100 | 2.5 | 67.1 |
| | | | | 0.17 | 100 | 0 | 75.7 |
| | | | | 2.5 | 100 | 0 | 81.2 |
| 14 | 145 | 0.5 | 10 | 0 | 100 | 1.5 | 81.6 |
| | | | | 0.17 | 100 | 0 | 85.2 |
| | | | | 2.5 | 100 | 0 | 88.2 |

Increasing the temperature to 125° C. (table 2, experiment 9) increases the selectivity of the formation of ethyl levulinate to 77.9%. It is noteworthy that the selectivity still increases over time, even when furfuryl alcohol and the intermediate 2-(ethoxymethyl) furan are converted completely. Without wishing to be bound by theory, it is assumed that further intermediates, which cannot be detected by GC-analysis, are formed but the reaction time was not sufficient to fully convert them to the target product. Lowering the concentration of furfuryl alcohol in the reaction mixture to 5 wt % (table 2, experiment 10) leads to an increased product selectivity of 82.3%. At 135° C. the same behavior can be observed: A lower concentration of furfuryl alcohol in the reaction mixture leads to increased ethyl levulinate selectivity of 91.4% (table 2, experiment 12). There is also a limit for the concentration of sulfuric acid in the reaction mixture. When the concentration of sulfuric acid is raised to 0.75 wt %, the ethyl levulinate selectivity drops to 81.2% (table 2, experiment 13).

The next step was to check the reaction outcome when increasing the average residence time in continuous mode to 0.75 hours (table 3). (The previous experiments were all concluded with an average residence time of 0.5 h.)

TABLE 3
Influence of increasing residence time to 0.75 h
on the formation of ethyl levulinate

| | | | | Time of | | Selectivity | Selectivity |
|---|---|---|---|---|---|---|---|
| | | | | sample | Conversion | 2-(ethoxy | ethyl |
| No. of | | H₂SO₄ | FFA | taking | of FFA | methyl)furan | levulinate |
| experiment | T [° C.] | [wt %] | [wt %] | [h] | [%] | [%] | [%] |
| 15 | 135 | 0.5 | 5 | 0 | 100 | 2.5 | 76.4 |
| | | | | 0.75 | 100 | 0 | 85.6 |
| | | | | 2.5 | 100 | 0 | 90.1 |
| 16 | 140 | 0.5 | 5 | 0 | 100 | 1.4 | 79.6 |
| | | | | 0.75 | 100 | 0 | 91.5 |
| | | | | 2.5 | 100 | 0 | 94.4 |
| 17 | 145 | 0.5 | 5 | 0 | 100 | 1.4 | 80.1 |
| | | | | 0.75 | 100 | 0 | 85.8 |
| | | | | 2.5 | 100 | 0 | 89.6 |

Increasing the residence time to 0.75 h improved the ethyl levulinate selectivity towards the end of the reaction. By a longer residence time, a lower local concentration of furfuryl alcohol in the reaction mixture is obtained, therefore side product formation is suppressed. The best results were obtained at 140° C. (table 3, experiment 16), at higher temperatures the product selectivity dropped again due to side product formation.

At 135° C., longer residence times of 1.0 h and 1.5 h were additionally tested (table 4). By increasing the residence time, the previously observed trend continues and higher dilution of the furfuryl alcohol leads to less side products. The limit of the selectivity is reached at 1.0 h or 1.5 h residence time in continuous mode, here the selectivity cannot be pushed higher than 94.5% or 94.2% respectively (table 4, experiments 18 and 19). With a residence time of 1.5 h the concentration of furfuryl alcohol in the reaction mixture was increased (table 4, experiments 20 to 22). Despite the higher concentration of 10 wt % furfuryl alcohol in the reaction mixture, 92.8% selectivity is obtained, and with 15 wt % of furfuryl alcohol, the selectivity only slightly drops to 91.2%.

TABLE 4
Influence of increasing the residence time and the concentration of furfuryl alcohol
in the reaction mixture on the formation of ethyl levulinate

| No. of | | | | Resi- | time of | Conver- | Selectivity | Selectivity |
|---|---|---|---|---|---|---|---|---|
| experi- | | H₂SO₄ | FFA | dence | sample | sion of | 2-(ethoxy- | ethyl |
| ment | T [° C.] | [wt %] | [wt %] | time [h] | taking [h] | FFA [%] | methyl) furan [%] | levulinate [%] |
| 18 | 135 | 0.5 | 5 | 1.0 | 0 | 100 | 1.2 | 85.9 |
| | | | | | 0.5 | 100 | 0 | 91.5 |
| | | | | | 1.5 | 100 | 0 | 94.5 |
| 19 | 135 | 0.5 | 5 | 1.5 | 0 | 100 | 0.8 | 85.7 |
| | | | | | 0.5 | 100 | 0 | 90.2 |
| | | | | | 1.5 | 100 | 0 | 94.2 |
| 20 | 135 | 0.5 | 7.5 | 1.5 | 0 | 100 | 0 | 86.0 |
| | | | | | 0.5 | 100 | 0 | 96.8 |
| | | | | | 1.5 | 100 | 0 | 92.1 |
| 21 | 135 | 0.5 | 10 | 1.5 | 0 | 100 | 0 | 83.2 |
| | | | | | 0.5 | 100 | 0 | 89.2 |
| | | | | | 1.5 | 100 | 0 | 92.8 |
| 22 | 135 | 0.5 | 15 | 1.5 | 0 | 100 | 0 | 85.5 |
| | | | | | 0.5 | 100 | 0 | 90.6 |
| | | | | | 1.5 | 100 | 0 | 91.2 |
| 23 | 135 | 0.5 | 5 | 1.5 | 0 | 100 | 0.8 | 73.9 |
| | | | | | 0.5 | 100 | 0 | 89.5 |
| | | | | | 1.5 | 100 | 0 | 92.1 |
| | | | | | 2.5 | 100 | 0 | 94.0 |

3. Formation of n-Butyl Ester of Levulinic Acid (n-Butyl Levulinate)

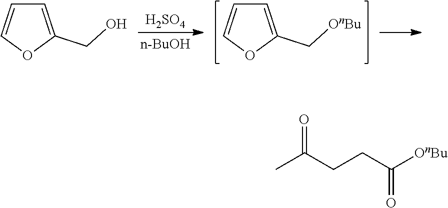

The formation of n-butyl levulinate was investigated using the stop-flow reactor and the procedure described above.

When the temperature is increased from 105° C. (table 5, experiments 24 to 25) to 115° C., the selectivity of n-butyl levulinate formation increases up to 64.4% (table 5, experiment 26). No improvement was observed when the reaction time was increased to 5 h. As observed for the formation of ethyl levulinate, decreasing the concentration of sulfuric acid in the reaction mixture leads to lower overall selectivity, because the reaction proceeds too slow and side product formation occurs (table 5, experiments 27 to 28).

TABLE 5
Influence of variation of the concentration of sulfuric acid and/or of FFA in the
reaction mixture at 105° C. and 115° C. on the formation of n-butyl
levulinate. Residence time in continuous mode: 0.5 hours.

| No. of | | | | time of | | Selectivity | Selectivity |
|---|---|---|---|---|---|---|---|
| experi- | | H₂SO₄ | FFA | sample | conv. of | 2-(n-butoxy- | n-butyl |
| ment | T [° C.] | [wt %] | [wt %] | taking [h] | FFA [%] | methyl) furan [%] | levulinate [%] |
| 24 | 105 | 0.5 | 10 | 0 | 97.6 | 16.1 | 32.1 |
| | | | | 2.5 | 100 | 0.1 | 57.9 |
| 25 | 105 | 0.5 | 5 | 0 | 97.8 | 17.7 | 29.4 |
| | | | | 2.5 | 100 | 0 | 48.0 |
| 26 | 115 | 0.5 | 10 | 0 | 99.3 | 8.9 | 48.3 |
| | | | | 0.17 | 100 | 5.9 | 54.4 |
| | | | | 2.5 | 100 | 0 | 64.4 |
| 27 | 115 | 0.1 | 10 | 0.75 | 100 | 15.5 | 43.5 |
| | | | | 2.5 | 100 | 3.3 | 56.6 |
| 28 | 115 | 0.05 | 10 | 0 | 78.7 | 23.7 | 25.3 |
| | | | | 1.5 | 100 | 15.7 | 43.4 |
| | | | | 2.5 | 100 | 9.3 | 50.8 |

The same behavior was observed at 125° C. (Table 6). When using a reaction mixture comprising 10 wt % furfuryl alcohol and 0.5 wt % sulfuric acid at 125° C., the n-butyl levulinate selectivity increased to 74.8% after 5 h stirring time (table 6, experiment 30). By lowering the concentration of sulfuric acid in the reaction mixture to 0.1 wt %, the overall selectivity of intermediate 2-(n-butoxymethyl) furan and butyl levulinate becomes lower.

TABLE 6
Influence of variation of concentration of sulfuric acid and/or of FFA in the
reaction mixture on the formation of n-butyl levulinate at a temperature of
125° C. Residence time in continuous mode: 0.5 hours.

| No. of | | | | time of | | Selectivity | Selectivity |
|---|---|---|---|---|---|---|---|
| experi- | | H₂SO₄ | FFA | sample | conv. of | 2-(n-butoxy- | n-butyl |
| ment | T [° C.] | [wt %] | [wt %] | taking [h] | FFA [%] | methyl)furan[%] | levulinate [%] |
| 29 | 125 | 0.5 | 10 | 0 | 98.9 | 12.1 | 53.5 |
| | | | | 0.08 | 100 | 9.6 | 58.6 |
| | | | | 1.0 | 100 | 0.1 | 72.5 |
| | | | | 2.5 | 100 | 0 | 73.1 |
| 30 | 125 | 0.5 | 10 | 5 | 100 | 0 | 74.8 |
| 31 | 125 | 0.1 | 10 | 0 | 81.1 | 23.5 | 27.9 |
| | | | | 0.75 | 100 | 15.2 | 44.0 |
| | | | | 2.5 | 100 | 2.3 | 58.6 |

By increasing the temperature to 135° C., a n-butyl levulinate selectivity of 83.7% was obtained after 2.5 h (table 7, experiment 32), and further increasing the temperature to 145° C. results in an increase of the selectivity to 88.8% (table 7, experiment 33). As in the case of ethyl levulinate, the selectivity increases over time even after substantially full conversion of furfuryl alcohol and the intermediate 2-(n-butoxymethyl) furan. Increasing the reaction temperature further to 155° C. led to a peak in selectivity after 1.0 h with 90.7%, but then decomposition occurred, and the selectivity dropped to 88.4% after 2.5 h (table 7, experiment 34).

TABLE 7
Influence of increased temperature on the formation of n-butyl levulinate.
Residence time in continuous mode: 0.5 hours.

| No. of | | | | time of | | Selectivity | Selectivity |
|---|---|---|---|---|---|---|---|
| experi- | | H₂SO₄ | FFA | sample | conv. of | 2-(n-butoxy- | n-butyl |
| ment | T [° C.] | [wt %] | [wt %] | taking [h] | FFA [%] | methyl)furan[%] | levulinate [%] |
| 32 | 135 | 0.5 | 10 | 0 | 100 | 5.9 | 65.2 |
| | | | | 0.75 | 100 | 0 | 80.7 |
| | | | | 2.5 | 100 | 0 | 83.7 |
| 33 | 145 | 0.5 | 10 | 0 | 100 | 3.3 | 79.7 |
| | | | | 0.5 | 100 | 0 | 86.0 |
| | | | | 2.5 | 100 | 0 | 88.8 |
| 34 | 155 | 0.5 | 10 | 0 | 100 | 2.3 | 81.6 |
| | | | | 1.0 | 100 | 0 | 90.7 |
| | | | | 2.5 | 100 | 0 | 88.4 |

As the results at 145° C. and 155° C. were very promising, further optimization around these conditions was investigated (table 8). At 145° C., with an increased concentration of sulfuric acid (0.75 wt %) and 10 wt % furfuryl alcohol in the reaction mixture, a n-butyl levulinate selectivity of 91.4% was achieved (table 8, experiment 35). With 0.5 wt % sulfuric acid and a 5 wt % furfuryl alcohol in the reaction mixture, the selectivity could be increased to 93.1% (table 8, experiment 36). Due to faster reaction and higher dilution (and therefore less polymerization tendency) an experiment at 155° C. using 0.75 wt % sulfuric acid and 5 wt % furfuryl alcohol in the reaction mixture leads to a peak selectivity of 98.4% after 0.75 h, but 10 then decomposition started and after 1.5 h the remaining n-butyl levulinate selectivity dropped to 96.9% (table 8, experiment 37).

TABLE 8
Influence of variation of concentration of sulfuric acid and of FFA in the reaction
mixture on the formation of n-butyl levulinate at a temperature of 145° C.
Residence time in continuous mode: 0.5 hours.

| No. of | | | | time of | | Selectivity | Selectivity |
|---|---|---|---|---|---|---|---|
| experi- | | H₂SO₄ | FFA | sample | conv. of | 2-(n-butoxy- | n-butyl |
| ment | T [° C.] | [wt %] | [wt %] | taking [h] | FFA [%] | methyl) furan [%] | levulinate [%] |
| 35 | 145 | 0.75 | 10 | 0 | 100 | 1.7 | 51.6 |
| | | | | 0.08 | 100 | 0.3 | 86.8 |
| | | | | 0.25 | 100 | 0 | 88.5 |
| | | | | 2.5 | 100 | 0 | 91.4 |
| 36 | 145 | 0.5 | 5 | 0.25 | 100 | 0 | 89.0 |
| | | | | 2.5 | 100 | 0 | 93.1 |
| 37 | 155 | 0.75 | 5 | 0 | 100 | 2.2 | 86.5 |
| | | | | 0.17 | 100 | 0 | 95.3 |
| | | | | 0.75 | 100 | 0 | 98.4 |
| | | | | 1.5 | 100 | 0 | 96.9 |

### 4. Formation of Methyl Ester of Levulinic Acid (Methyl Levulinate)

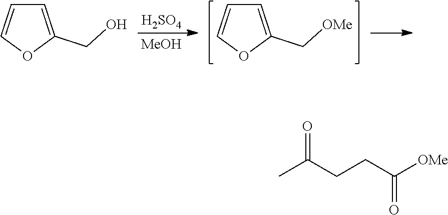

The formation of methyl levulinate was investigated using the stop-flow reactor and the procedure described above.

In a first step using 10 wt % furfuryl alcohol in the reaction mixture, the optimal reaction temperature was determined (table 9). Temperatures of 120° C. and 130° C. (table 9, experiments 38 to 39) lead to moderate methyl levulinate selectivity of 75 to 76%. Similar to the previous experiments the only intermediate observed by GC is 2-(methoxymethyl) furan which reacts quickly to consecutive intermediates, which are not detected, but then reacts to the target product after additional reaction time. The best results were obtained at 140° C. (table 9, experiment 40) with a selectivity of 86.9%. Increasing the temperature to 150° C. results in a lower selectivity, due to formation of side product and/or decomposition of the target product (table 9, experiment 41).

TABLE 9
Influence of temperature on the formation of methyl levulinate. Residence time in
continuous mode: 0.5 hours.

| No. of | | | | time of | | Selectivity | Selectivity |
|---|---|---|---|---|---|---|---|
| experi- | | H₂SO₄ | FFA | sample | conv. of | 2-(methoxy- | methyl |
| ment | T [° C.] | [wt %] | [wt %] | taking [h] | FFA [%] | methyl) furan [%] | levulinate [%] |
| 38 | 120 | 0.5 | 10 | 0 | 99.7 | 0 | 56.5 |
| | | | | 0.75 | 100 | 0 | 74.9 |
| | | | | 2.0 | 100 | 0 | 75.8 |
| 39 | 130 | 0.5 | 10 | 0 | 99.8 | 0.7 | 62.7 |
| | | | | 0.75 | 100 | 0 | 72.9 |
| | | | | 2.5 | 100 | 0 | 75.2 |
| 40 | 140 | 0.5 | 10 | 0 | 100 | 1.4 | 73.1 |
| | | | | 0.75 | 100 | 0 | 82.4 |
| | | | | 2.5 | 100 | 0 | 86.9 |
| 41 | 150 | 0.5 | 10 | 0 | 100 | 0.7 | 74.7 |
| | | | | 0.75 | 100 | 0 | 82.5 |
| | | | | 2.0 | 100 | 0 | 84.1 |

At 140° C., the influence of an increased residence time was investigated (table 10). As observed for the formation of ethyl levulinate and n-butyl levulinate, longer residence time leads to a lower concentration of furfuryl alcohol in the reaction mixture, less side product formation and therefore higher selectivity after additional reaction time. By increasing the residence time to 1.5 h the selectivity could be increased to 95.8% (table 10, experiment 43). With high residence time, it is possible to increase the concentration of furfuryl alcohol in the reaction mixture to 15% (table 10, experiment 45), with the obtained selectivity being >91%.

TABLE 10
Influence of increasing residence time and of the concentration of
furfuryl alcohol in the reaction mixture on the formation
of methyl levulinate at a temperature of 140° C.

| | | | | | | | Selectivity | Selectivity |
|---|---|---|---|---|---|---|---|---|
| No. of | | | | Resi- | time of | Conver- | 2-(methoxy- | methyl |
| experi- | | H₂SO₄ | FFA | dence | sample | sion of | methyl) | levulinate |
| ment | T [° C.] | [wt %] | [wt %] | time [h] | taking [h] | FFA [%] | furan [%] | [%] |
| 42 | 140 | 0.5 | 5 | 0.5 | 0 | 99.6 | 0 | 62.8 |
| | | | | | 0.5 | 100 | 0 | 88.6 |
| | | | | | 2.0 | 100 | 0 | 90.1 |
| 43 | 140 | 0.5 | 5 | 1.5 | 0 | 99.2 | 0 | 89.0 |
| | | | | | 0.25 | 100 | 0 | 95.8 |
| | | | | | 1.0 | 100 | 0 | 95.8 |
| 44 | 140 | 0.5 | 10 | 1.5 | 0 | 99.8 | 0 | 69.8 |
| | | | | | 0.25 | 100 | 0 | 87.6 |
| | | | | | 0.75 | 100 | 0 | 93.8 |
| | | | | | 1.5 | 100 | 0 | 95.0 |
| 45 | 140 | 0.5 | 15 | 1.5 | 0 | 99.8 | 0 | 68.9 |
| | | | | | 0.25 | 100 | 0 | 84.5 |
| | | | | | 1.25 | 100 | 0 | 91.8 |

### 5. Improved Reactor System for Continuous Operation

In an improved reactor system, a residence time section in the form of a 60 mL capillary tube is arranged downstream of the autoclave, in order to provide favorable conditions for the conversion of the intermediate formed in the autoclave to the target product. In the improved reactor system, the reaction takes place in a 60 mL nickel alloy autoclave. The autoclave is in fluid connection with two independently operated supply lines under automated control and equipped with electronic balances to obtain a mass balance of the reaction. The first supply line supplies a first supply flux comprising furfuryl alcohol and aliphatic alcohol, the second line supplies a second supply flux comprising sulfuric protic acid and an aliphatic alcohol which is the same as in the first supply flux. The supply rates are adjusted by two pumps independently, so that the average residence time in the autoclave and the concentration of sulfuric acid in the autoclave can be adjusted. The autoclave is equipped with a thermostat for temperature control, a stirrer and flow breaker. A high stirring speed of 1000 rpm is used to guarantee substantially complete mixing and fast dilution of the added furfuryl alcohol. The reaction was run for at least 5 average residence times (achieving steady state and a >99% exchange of the reaction mixture) before samples were taken from system.

The autoclave is equipped with a sampling outlet and an exit to the residence time section. The latter is designed as capillary tube. The exit of the capillary tube is equipped with an exit to a needle valve, which is controlled by pressure (set to the vapor pressure of the used aliphatic alcohol at the target temperature).

The improved reactor system was used to investigate the formation of ethyl levulinate at 135° C. (experiment 46) and 140° C. (experiment 47) with a reaction mixture comprising 15 wt % furfuryl alcohol and 0.5 wt % sulfuric acid at a residence time of 1.5 hours in the autoclave. Samples were taken upstream and downstream of the capillary tube and analyzed by gas chromatography (GC) using 1-methoxy-2-(2-methoxyethoxy) ethane (also known as diglyme or DEGDME) as internal standard. The obtained wt % were then used to calculate furfuryl alcohol conversion and selectivity with respect to the formation of the intermediate 2-(alkoxymethyl) furan and of the target product (alkyl ester of levulinic acid).

In experiment 46, for both samples the conversion of furfuryl alcohol was 100%. Also in experiment 47, for both samples the conversion of furfuryl alcohol was 100%.

TABLE 11
Formation of ethyl levulinate in continuous operation at a temperature of 135°
C. (experiment 46) and 140° C. (experiment 47), residence time of 1.5 h in the autoclave.

| | Sample upstream | Sample upstream | Sample down- | Sample down- |
|---|---|---|---|---|
| | of capillary tube - | of capillary tube - | stream of capillary | stream of capillary |
| | Selectivity | Selectivity ethyl | tube - Selectivity | tube - Selectivity |
| No. of | 2-(ethoxymethyl)furan | levulinate | 2-(ethoxymethyl)furan | ethyl levulinate |
| experiment | [%] | [%] | [%] | [%] |
| 46 | 0.5 | 85.9 | 0 | 96.6 |
| 47 | 0.3 | 93.0 | 0 | 97.2 |

In continuous mode at 135° C., a selectivity of 85.9% of ethyl levulinate was achieved upstream of the capillary tube, with a selectivity of 2-(ethoxymethyl) furan of 0.5%. Similar to the previous experiments the only intermediate observed by GC is 2-(ethoxymethyl) furan which reacts quickly to consecutive intermediates, which are not detected, but then react to the target product after additional reaction time. The sample taking after the residence time section (capillary tube) showed a selectivity of ethyl levulinate of 96.6% (Table 11, experiment 46). When the temperature was increased from 135° C. to 140° C., a selectivity of 93.0% of ethyl levulinate was achieved upstream of the capillary tube, with a selectivity of 2-(ethoxymethyl) furan of 0.3%. The sample taking after the residence time section (capillary tube) showed a selectivity of ethyl levulinate of 97.2% (Table 11, experiment 47).

### 6. Larger Reactor System in Continuous Operation

In the larger reactor system, the reaction takes place in a 2500 mL nickel alloy autoclave, with a filling level of 1800 mL. The autoclave is in fluid connection with two independently operated supply lines under automated control and equipped with electronic balances to obtain a mass balance of the reaction. The first supply line supplies a first supply flux comprising furfuryl alcohol and aliphatic alcohol, the second line supplies a second supply flux comprising a protic acid, and an aliphatic alcohol which is the same as in the first supply flux. The supply rates are adjusted by two pumps independently, so that the average residence time in the autoclave and the concentration of protic acid in the autoclave can be adjusted. The autoclave is equipped with a thermostat for temperature control, a stirrer and flow breaker. A high stirring speed of 800 rpm is used to guarantee substantially complete mixing and fast dilution of the added furfuryl alcohol. No residence time section is arranged downstream of the autoclave.

The autoclave is equipped with a sampling outlet. The exit of the autoclave is equipped with an exit to a needle valve, which is controlled by mass flow (to keep the filling level in the autoclave at 1800 mL).

The reaction was run for at least 5 average residence times (achieving steady state and a >99% exchange of the reaction mixture) before samples were taken from system. Samples were taken from the exit of the autoclave and analyzed by gas chromatography (GC) using 1-methoxy-2-(2-methoxyethoxy) ethane (also known as diglyme or DEGDME) as internal standard. The obtained wt % were then used to calculate furfuryl alcohol conversion and selectivity with respect to the formation of the intermediate 2-(alkoxymethyl) furan and of the target product (alkyl ester of levulinic acid).

This reactor system was used to investigate the influence of the selection of the protic acids (cf. table 12) and to investigate the influence of higher concentrations of furfuryl alcohol in aliphatic alcohol in the first supply flux (table 13).

TABLE 12
Influence of the selection of the protic acid on the formation
of ethyl levulinate in continuous operation at a temperature
of 135° C. Residence time in the autoclave: 1.5 h.

| | | | Selectivity | Selectivity |
|---|---|---|---|---|
| | FFA [wt %] | | 2-(ethoxy- | ethyl |
| No. of | in first | Protic acid | methyl)furan | levulinate |
| experiment | supply flux | [wt %] | [%] | [%] |
| 48 | 15 | H₂SO₄ [0.5] | 1.0 | 91.0 |
| 49 | 15 | MsOH [0.5] | 3.0 | 84.0 |
| 50 | 15 | p-TsOH [0.8] | 1.7 | 89.6 |

The reactor system was used to investigate the formation of ethyl levulinate at 135° C. with a first supply flux comprising 15 wt % furfuryl alcohol. The weight fraction of the protic acid in the second supply flux was adjusted so that approximately the same molar ratio of protic acid to furfuryl alcohol is obtained. The aliphatic alcohol is ethanol.

For all taken samples the conversion of furfuryl alcohol was 100%.

Using sulfuric acid (H₂SO₄) a selectivity of 91.0% of ethyl levulinate was achieved, with a selectivity of 2-(ethoxymethyl) furan of 1.0% (Table 12, experiment 48). Using methanesulfonic acid (MsOH) a selectivity of 84.0% of ethyl levulinate was achieved, with a selectivity of 2-(ethoxymethyl) furan of 3.0% (Table 12, experiment 49). Using para-toluenesulfonic acid (p-TsOH) a selectivity of 89.6% of ethyl levulinate was achieved, with a selectivity of 2-(ethoxymethyl) furan of 1.7% (Table 12, experiment 50). The highest selectivity of ethyl levulinate was obtained with sulfuric acid, which is therefore the preferred protic acid.

To enable application of higher furfuryl alcohol concentrations in the first supply flux, the first supply line (which supplies a first supply flux comprising furfuryl alcohol and aliphatic alcohol) was split close to its entry into the autoclave to allow supplying the first supply flux via two inlets into the autoclave. Using this setup, the formation of ethyl levulinate resp. methyl levulinate was investigated at 135° C. resp. 140° C. using a first supply flux comprising 25 wt % furfuryl alcohol. The concentration of sulfuric acid was 0.83 wt %. The residence time in the autoclave was 1.5 hours. Samples were taken and analyzed by gas chromatography (GC) using 1-methoxy-2-(2-methoxyethoxy) ethane (also known as diglyme or DEGDME) as internal standard. The obtained wt % were then used to calculate furfuryl alcohol conversion and selectivity with respect to the formation of the intermediate 2-(alkoxymethyl) furan and of the target product (alkyl ester of levulinic acid).

TABLE 13
Formation of alkyl levulinate in continuous operation at given
temperature, residence time of 1.5 h in the autoclave.

| | | | | Selectivity | Selectivity |
|---|---|---|---|---|---|
| | | FFA [wt %] | | 2-(alkoxy- | alkyl |
| No. of | T | in first | Aliphatic | methyl)furan | levulinate |
| experiment | [° C.] | supply flux | alcohol | [%] | [%] |
| 51 | 135 | 25 | Ethanol | 0.5 | 91.4 |
| 52 | 140 | 25 | Methanol | 0.1 | 84.5 |

For all taken samples the conversion of furfuryl alcohol was 100%.

In continuous mode at 135° C. using ethanol as aliphatic alcohol, a selectivity of 91.4% of ethyl levulinate was achieved, with a selectivity of 2-(ethoxymethyl) furan of 0.5% (Table 13, experiment 51). Using methanol as aliphatic alcohol, a selectivity of 84.5% of methyl levulinate was achieved, with a selectivity of 2-(methoxymethyl) furan of 0.1% (Table 13, experiment 52).

Irrespective of the high concentration of furfuryl alcohol in the first supply flux, a high selectivity for the target alkyl levulinate is achieved. Accordingly, due to the fast dilution of the furfuryl alcohol in the reaction mixture resulting from contacting the first and the second supply flux in the pressurized continuous flow reactor system, undesired polymerization of furfuryl alcohol is avoided.

1. A process for forming an alkyl ester of levulinic acid or one or more reaction products thereof,
said process comprising at least the following steps
(i) preparing or providing
a first supply flux comprising furfuryl alcohol and aliphatic alcohol, and
a second supply flux comprising at least one protic acid, wherein said protic acid is selected from the group consisting of sulfuric acid, halogen sulfonic acids, aliphatic sulfonic acids, aromatic sulfonic acids and alkyl-aromatic sulfonic acids,
(ii) supplying the first supply flux and the second supply flux prepared or provided in step (i) to a pressurized continuous flow reactor system and contacting the first supply flux and the second supply flux in said pressurized continuous flow reactor system so that in the resulting reaction mixture alkyl ester of levulinic acid is formed by a protic acid-catalyzed reaction of furfuryl alcohol with said aliphatic alcohol,
wherein the second supply flux supplies 2.6*10⁻² moles to 9*10⁻¹ moles of protic acid per mole of furfuryl alcohol supplied by the first supply flux,
wherein the ratio of the total molar amount of aliphatic alcohol supplied to the pressurized continuous flow reactor system to the total molar amount of alkyl ester of levulinic acid supplied to the pressurized continuous flow reactor system is at least 100:1.

2. The process according to claim 1, wherein
said aliphatic alcohol in the first supply flux is selected from the group consisting of methanol, ethanol, n-propanol, i-propanol, n-butanol and mixtures thereof,
and/or
said alkyl ester of levulinic acid is selected from the group consisting of methyl levulinate, ethyl levulinate, n-propyl levulinate, i-propyl levulinate, n-butyl levulinate and mixtures thereof.

3. The process according to claim 1, wherein said protic acid is sulfuric acid.

4. The process according to claim 1, wherein one or more parameters from the group consisting of
the molar ratio of protic acid supplied by the second supply flux relative to furfuryl alcohol supplied by the first supply flux,
the concentration of furfuryl alcohol in the reaction mixture resulting from contacting the first and the second supply flux in the pressurized continuous flow reactor system,
the concentration of protic acid in the reaction mixture resulting from contacting the first and the second supply flux in the pressurized continuous flow reactor system,
the temperature in the pressurized continuous flow reactor system, and
the residence time in the pressurized continuous flow reactor system
are adjusted so that the selectivity of the formation of alkyl esters of levulinic acid is 70% or more.

5. The process according to claim 1, wherein
the concentration of furfuryl alcohol in the reaction mixture resulting from contacting the first and the second supply flux in the pressurized continuous flow reactor system is less than 15 wt %,
and/or
the concentration of protic acid in the reaction mixture resulting from contacting the first and the second supply flux in the pressurized continuous flow reactor system is in the range of from 0.1 wt % to 1.5 wt %,
and/or
the temperature in the pressurized continuous flow reactor system is in the range of from 105° C. to 160° C.
and/or
the residence time in the pressurized continuous flow reactor system is in the range of from 0.25 to 5.0 hours.

6. The process according to claim 1, wherein no compound selected from the group consisting of compounds of Bi, Ga, Al, Sn and Fe, is present in the reaction mixture.

7. The process according to claim 1, wherein said pressurized continuous flow reactor system comprises a material selected from the group consisting of stainless steel and nickel alloys.

8. The process according to claim 1, wherein non-reacted aliphatic alcohol is separated from the product flux leaving the pressurized continuous flow reactor system, and said non-reacted aliphatic alcohol is recycled to said pressurized continuous flow reactor system wherein alkyl ester of levulinic acid is formed by a protic acid-catalyzed reaction of furfuryl alcohol with said aliphatic alcohol.

9. The process according to claim 1, further comprising the step of
(iii) chemically converting the alkyl ester of levulinic acid formed in step (ii) into one or more reaction products, wherein chemically converting said alkyl ester of levulinic acid into one or more reaction products comprises elimination of aliphatic alcohol,
wherein said one or more reaction products are selected from the group consisting of levulinic acid, 1,4-pentanediol, gamma-valerolactone, alpha-angelicalactone, beta-angelicalactone and 2-methyl-tetrahydrofuran,
wherein optionally aliphatic alcohol eliminated in step (iii) is recycled to said pressurized continuous flow reactor system wherein alkyl ester of levulinic acid is formed by a protic acid-catalyzed reaction of furfuryl alcohol with said aliphatic alcohol.

10. The process according to claim 1, wherein
no compound selected from the group consisting of levulinic acid and alkyl esters of levulinic acid is supplied to the pressurized continuous flow reactor system.

11. A method of using a pressurized continuous flow reactor system, the method comprising using the pressurized continuous flow reactor system for forming an alkyl ester of levulinic acid by a protic acid-catalyzed reaction of furfuryl alcohol with aliphatic alcohol by the process according to claim 1.

12. (canceled)

13. (canceled)

14. (canceled)

15. (canceled)

16. The process for forming alkyl ester of levulinic acid or one or more reaction products thereof according to claim 1, wherein the step (ii) the second supply flux supplies 3*10⁻² moles to 5*10⁻¹ moles of protic acid per mole of furfuryl alcohol supplied by the first supply flux.

17. The process for forming alkyl ester of levulinic acid or one or more reaction products thereof according to claim 1, wherein the step (ii) the second supply flux supplies 3*10⁻² moles to 3*10⁻¹ moles of protic acid per mole of furfuryl alcohol supplied by the first supply flux.

18. The process for forming alkyl ester of levulinic acid or one or more reaction products thereof according to claim 1, wherein the step (ii) the second supply flux supplies 3*10⁻² moles to 7*10⁻² moles of protic acid per mole of furfuryl alcohol supplied by the first supply flux.

19. The process according to claim 4, wherein
the temperature in the pressurized continuous flow reactor system and
the residence time in the pressurized continuous flow reactor system
are adjusted so that the selectivity of the formation of alkyl esters of levulinic acid is 80% or more.

20. The process according to claim 1, wherein
the concentration of furfuryl alcohol in the reaction mixture resulting from contacting the first and the second supply flux in the pressurized continuous flow reactor system is less than 10 wt %,
and/or
the concentration of protic acid in the reaction mixture resulting from contacting the first and the second supply flux in the pressurized continuous flow reactor system is in the range of from 0.1 wt % to 1.5 wt %,
and/or
the temperature in the pressurized continuous flow reactor system is in the range of from 115° C. to 145° C.
and/or
the residence time in the pressurized continuous flow reactor system is in the range of from 0.5 to 3.0 hours.

21. The process according to claim 1, wherein
the concentration of furfuryl alcohol in the reaction mixture resulting from contacting the first and the second supply flux in the pressurized continuous flow reactor system is less than 5 wt %,
and/or
the concentration of protic acid in the reaction mixture resulting from contacting the first and the second supply flux in the pressurized continuous flow reactor system is in the range of from 0.1 wt % to 1.5 wt %,
and/or
the temperature in the pressurized continuous flow reactor system is in the range of from 115° C. to 145° C.,
and/or
the residence time in the pressurized continuous flow reactor system is in the range of from 0.5 to 3.0 hours.
